(19) United States
(12) Patent Application Publication
(10) Pub. No.: US 2024/0360521 A1
(43) Pub. Date: Oct. 31, 2024

(54) MICROBIOME DIAGNOSTICS AND THE DETECTION OF MICROBIAL STRANDS

(71) Applicant: CARDIAI TECHNOLOGIES, Calgary (CA)

(72) Inventors: Minal Borkar Tripathi, Calgary (CA); Sidharth Singh Bhinder, Calgary (CA); Anmol Singh Kapoor, Calgary (CA); Amrita Kaur, Calgary (CA)

(21) Appl. No.: 18/647,007

(22) Filed: Apr. 26, 2024

Related U.S. Application Data
(60) Provisional application No. 63/498,627, filed on Apr. 27, 2023.

(51) Int. Cl. C12Q 1/6888; C12Q 1/6844; G01N 33/569

(52) U.S. Cl. CPC C12Q 1/6888; C12Q 1/6844; G01N 33/569; G01N 2469/20

(57) ABSTRACT

The present disclosure relates to systems and methods for for diagnosing medical conditions using microbiome diagnostics by collecting a sample from a patient, isolating microbial strains from the sample by placing the sample on a culture medium and incubating, analyzing the isolated microbial strains to detect the presence of certain pathogens using polymerase chain reaction (PCR) or other suitable analytic methods to detect the presence of pathogens including bacteria, viruses, fungi, or other microbes, interpreting the results to determine if the patient has a medical condition and providing a diagnosis based on the interpretation of the results.

### CROSS-REFERENCE TO RELATED APPLICATIONS

This application claims the benefit of U.S. Provisional Application No. 63/498,627 titled “MICROBIOME DIAGNOSTICS AND THE DETECTION OF MICROBIAL STRANDS” filed by the applicant on Apr. 27, 2023, which is incorporated herein by reference in its entirety.

### FIELD OF THE INVENTION

Embodiments of the present invention relate to the field of microbiome diagnostics and more specifically methods for detecting microbial strains in order to diagnose medical conditions,

### DESCRIPTION OF THE RELATED ART

A microbiome can include an ecological community of commensal, symbiotic, and pathogenic microorganisms that are associated with an organism. Characterization of the human microbiome is a complex process. The human microbiome includes over 10 times more microbial cells than human cells, but characterization of the human microbiome is still in nascent stages such as due to limitations in sample processing techniques, genetic analysis techniques, and resources for processing large amounts of data.

A typical healthy individual's body is inhabited with trillions of microbes across various body sites called microbiomes. Some examples of microbiome sites include skin, intestinal, stomach, gut, oral, conjunctival, and vaginal. To better understand the role of these microbiomes and how they affect physiology and disease state we can analyze what microbes comprise a microbiome and how they correlate or affect the health status and clinical response of an individual. For example, the human gut microbiome is known to play a key role in many health conditions, including obesity, gastrointestinal health, nutrient absorption, and drug metabolism among others.

Present knowledge has clearly established the role of microbiome associations with multiple health conditions, and has become an increasingly appreciated mediator of host genetic and environmental factors on human disease development.

The microbiome is suspected to play at least a partial role in a number of health/disease-related states (e.g., preparation for childbirth, diabetes, auto-immune disorders, gastrointestinal disorders, rheumatoid disorders, neurological disorders, etc.). Further, the microbiome plays an essential role in human health, with dysbiosis of the microbiome linked to a range of diseases, including inflammatory bowel disease, diabetes, and obesity. Furthermore, the microbiome may mediate effects of environmental factors on human, plant, and/or animal health. Given the profound implications of the microbiome in affecting a subject's health, efforts related to the characterization of the microbiome, the generation of insights from the characterization, and the generation of therapeutics configured to rectify states of dysbiosis should be pursued.

The most commonly used methods for microbiome modulation involve antibiotics and probiotics. However, these methods are limited in their effectiveness and often result in unwanted side effects.

Further, the current methods and systems for analyzing the microbiomes of humans and/or providing therapeutic measures based on gained insights have, however, left many questions unanswered. Thus, there is a need in the field of microbiology for a new and useful method and/or system for modulating the microbiome to promote health and/or prevent diseases, microorganism-related health conditions and/or the associated relationships (e.g., specific features associated with microorganisms and/or conditions, etc.), such as for individualized and/or population-wide use. Therefore, the present invention provides a system for detecting and assessing microbiome diseases and an alternative method of microbiome modulation.

### SUMMARY OF THE INVENTION

Embodiments of the present invention relate to a method for diagnosing medical conditions using microbiome diagnostics, comprising:
a. Collecting a sample from a patient, wherein the sample is obtained from a biological source selected from the group consisting of blood, skin swab, urine, and feces;
b. Isolating microbial strains from the sample by placing the sample on a culture medium and incubating;
c. Analyzing the isolated microbial strains to detect the presence of certain pathogens using polymerase chain reaction (PCR) or other suitable analytic methods to detect the presence of pathogens including bacteria, viruses, fungi, or other microbes;
d. Interpreting the results to determine if the patient has a medical condition; and
e. Providing a diagnosis based on the interpretation of the results to an individual or a healthcare professional.

Another embodiment of the present invention relates to a system for diagnosing medical conditions using microbiome diagnostics, comprising:
a. means for collecting a sample from a patient, wherein the sample is obtained from a biological source selected from the group consisting of blood, skin swab, urine, and feces;
b. a means for isolating microbial strains from the sample by placing the sample on a culture medium and incubating;
C. a means for analyzing the isolated microbial strains to detect the presence of certain pathogens using polymerase chain reaction (PCR) or other suitable analytic methods to detect the presence of pathogens including bacteria, viruses, fungi, or other microbes;
d. a means for interpreting the results to determine if the patient has a medical condition, and
e. a means for providing a diagnosis based on the interpretation of the results by an individual or a healthcare professional.

In accordance with an embodiment of the present invention, the sample is a bodily fluid and is selected from a group consisting of saliva, urine, stool, or tissue samples from organs or other body parts.

In accordance with an embodiment of the present invention, the method also comprises a step of performing a sequencing test that sequences an entire genome or a portion of a genome of the microorganism to identify the presence of specific genes associated with a microbiome disease.

In accordance with an embodiment of the present invention, the PCR-based test amplifies a specific region of the microorganism's genome to detect the presence of specific DNA sequences associated with a microbiome disease.

In accordance with an embodiment of the present invention, the method includes a step of measuring the presence of specific antibodies associated with a microbiome disease.

In accordance with an embodiment of the present invention, presence of specific antibodies associated with the microbiome disease is measured by ELISA-based test.

In accordance with an embodiment of the present invention, the method further includes measuring presence of specific metabolites associated with the microbiome disease.

In accordance with an embodiment of the present invention, the presence of specific metabolites associated with the microbiome disease is measured by mass spectrometry-based test measures

In accordance with an embodiment of the present invention, the report includes a diagnosis of the presence of a microbiome disease and/or a treatment recommendation based on the results of said tests.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart that depicts an exemplary computer system for implementing a method described herein.

FIG. 2 is a flowchart illustrating a method for diagnosing medical conditions using microbiome diagnostics.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practised are described in sufficient detail to enable those skilled in the art to practice the disclosed embodiments. However, it will be obvious to a person skilled in the art that the embodiments of the invention may be practised with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention:

The term strains and strains are used interchangeably for convenience throughout the draft.

The term “microbiome”, as used herein, refers to the ecological community of commensal, symbiotic, or pathogenic microorganisms that inhabit a body space on a subject.

The terms “determining”, “measuring”, “evaluating”, “assessing,” “assaying,” and “analyzing” can be used interchangeably herein to refer to any form of measurement, and include determining if an element is present or not. (e.g., detection). These terms can include both quantitative and/or qualitative determinations. Assessing may be relative or absolute.

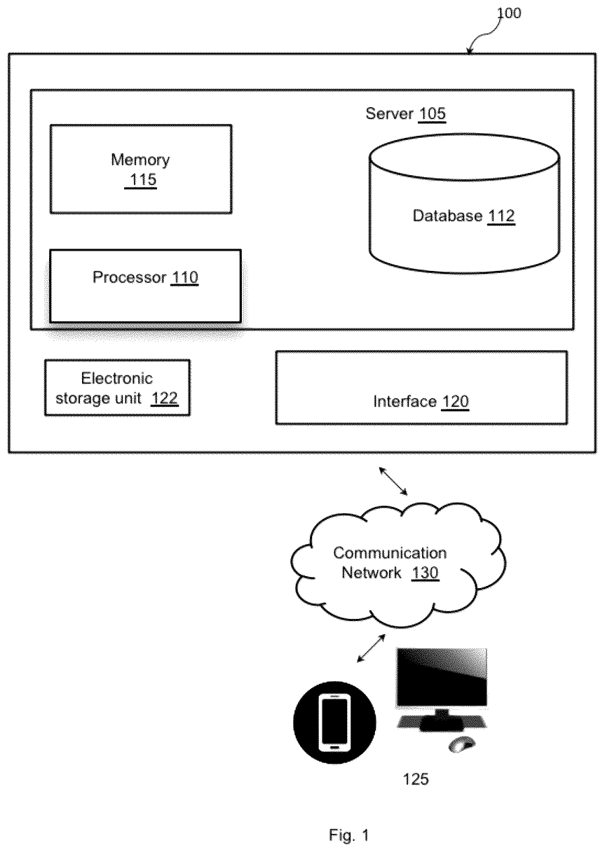

FIG. 1 depicts an exemplary computer system for implementing a method described herein. This includes a continually enlarging database the methods described herein allow this to be expanded in a cost-effective manner that hasn't been previously available.

The current disclosure provides computer systems for implementing any of the methods described herein. A computer system may be used to implement steps of, providing the biological sample containing microorganisms, for isolating the microorganisms from the biological sample, performing one or more tests on the isolated microorganisms and generating and providing a report based on results of the tests performed to a receiver.

For example, provided herein are computer systems for detecting the presence or absence of microbes. Also provided herein are computer systems for detecting a presence or absence of bacteria, fungi, archaea or other elements that comprise and maintain a microbiome.

The exemplary computer system 100 is adapted to implement a method described herein. The system 100 includes a server 105 programmed to implement exemplary methods described herein. The server 105 further includes a processor unit 110 which can be a single core processor, a multi core processor, or plurality of processors for parallel processing, a database 112, a memory 115 (e.g. random access memory, read-only memory, flash memory); an electronic storage unit 120 (e.g. hard disk); a user device 125 with communications interface for communicating with one or more other systems; and peripheral devices (not shown) which may include cache, other memory, data storage, and/or electronic display adaptors.

The memory 115, database 112, interface 120, and user devices 125 are in communication with the processing unit 110 through a communication interface including but not limited to wired networks such as a communication bus (solid lines), a motherboard and/or wireless network.

The database 112 can be a data storage unit for storing data. The database 112 can store files, such as subject reports, and/or communications with the subjects or users, data about subjects or users, such as, for example medical history, family history, demographic data and/or other clinical or personal information of potential relevance to a particular application, or any aspect of data associated with the invention.

The server 105 is operatively coupled to a computer network (“network”) 130 with the aid of the communications interface. The network 130 can be the Internet, an intranet and/or an extranet, an intranet and/or extranet that is in communication with the Internet, a telecommunication or data network. The network 130 in some cases, with the aid of the server 101, can implement a peer-to-peer network, which may enable devices coupled to the server 101 to behave as a client or a server.

The server 105 can communicate with one or more remote computer systems through the network 130. The one or more remote computer systems may be, for example, personal computers, laptops, tablets, telephones, Smart phones, or personal digital assistants. In some applications the computer system 100 includes a single server 101. In other situations, the system includes multiple servers in communication with one another through an intranet, extranet and/or the internet.

Methods as described herein can be implemented by way of machine (or computer processor) executable code (or software) stored on an electronic storage location of the server 105. During use, the code can be executed by the processing unit 110. In some cases, the code can be retrieved from the database 112 and stored on the memory 120 for ready access by the processing unit 110.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

In an embodiment, the computer system consists: a memory unit for receiving data comprising reports and results of samples of the subject; and computer-executable instructions for determining a presence or absence of at least disease in the subject based upon said analyzing.

In some embodiments, the system further comprises computer-executable instructions to generate a report of the presence or absence of at least one disease in the subject.

The system for detecting and assessing microbiome diseases by assaying a sample of a subject also comprises
a means for collecting a sample from a patient,
a means for isolating microbial strains from the sample by placing the sample on a culture medium and incubating;
a means for analyzing the isolated microbial strains to detect the presence of certain pathogens using polymerase chain reaction (PCR) or other suitable analytic methods to detect the presence of pathogens including bacteria, viruses, fungi, or other microbes;
a means for interpreting the results to determine if the patient has a medical condition, and
a means for providing a diagnosis based on the interpretation of the results by an individual or a healthcare professional.

In accordance with an embodiment of the present invention, the sample is obtained from a biological source selected from the group consisting but not limited to blood, skin swab, urine, feces, saliva, urine, stool, or tissue samples from organs or other body parts.

In alternative embodiment, the sample is a biological sample that can be any sample type from any microbiome on the body of a subject. Some examples of microbiomes that can be used with the present disclosure include the skin microbiome, umbilical microbiome, vaginal microbiome, conjunctival microbiome, intestinal microbiome, stomach microbiome, gut microbiome and oral microbiome, nasal microbiome, gastrointestinal tract microbiome, and the urogenital tract microbiome. Depending on the application the biological sample can be whole blood, serum, plasma, mucosa, saliva, cheek swab, urine, stool, cells, tissue, bodily fluid or a combination thereof.

In accordance with an embodiment of the present invention, the means of providing the sample could be a collection kit provided to the subject or a sampling apparatus integrated into the system.

Alternatively, means of providing the sample could be a simple swab or bodily fluid collection device.

Alternatively, means of providing the sample could be coupled with telemedicine infrastructure, allowing remote sample collection under guidance from healthcare professionals.

Further, sample collection could be automated or semi-automated, with built-in mechanisms for ensuring sample integrity.

Means for isolating microorganisms/microbial strains might include centrifugation, filtration, or other separation techniques. Alternatively, means for Isolation of microorganisms might involve miniaturized or rapid separation techniques suitable for point-of-care settings. Alternatively, microorganism isolation could be achieved through integrated microfluidic devices or other miniaturized techniques.

In accordance with an embodiment of the present invention, means for performing one or more tests on the isolated microorganisms selected from the group consisting of a sequencing test, a PCR-based test, an ELISA-based test, a mass spectrometry-based test, or other suitable tests to detect and identify the presence of a microbiome disease.

The PCR-based test amplifies a specific region of the microorganism's genome to detect the presence of specific DNA sequences associated with a microbiome disease.

The PCR-based assays for targeting specific microbial genes or regions. PCR-based tests may involve polymerase chain reaction (PCR) techniques targeting specific DNA sequences associated with known microbiome diseases. Primers designed for detecting pathogens like bacteria, viruses, or fungi could be employed

ELISA-based test for measuring the presence of specific antibodies associated with a microbiome disease.

ELISA assays detect microbial antigens or antibodies, mass spectrometry for proteomic analysis.ELISA-based tests may utilize enzyme-linked immunosorbent assays (ELISA) to detect antigens or antibodies indicative of microbiome diseases. Specific antibodies against target microorganisms or their toxins could be employed for detection.

Mass spectrometry-based test measures presence of specific metabolites associated with the microbiome disease.

Mass spectrometry-based tests may involve matrix-assisted laser desorption/ionization time-of-flight (MALDI-TOF) or other mass spectrometry techniques for identifying microorganisms based on their unique protein profiles.

Other suitable tests could include immunohistochemistry, flow cytometry, or culture-based methods for microbiome disease detection, depending on the nature of the microorganisms and the disease being assessed.

Testing methods could be streamlined versions of laboratory techniques, designed for rapid and simple execution, such as portable PCR devices or lateral flow assays.

Testing methods could include multiplexed assays capable of detecting multiple pathogens simultaneously, enhancing diagnostic efficiency.

Alternatively, for performing sequencing tests, the system may utilize next-generation sequencing (NGS) technologies such as Illumina sequencing, Oxford Nanopore sequencing, or other sequencing platforms to analyze the genetic composition of the isolated microorganisms.

In accordance with an embodiment of the present invention, means for interpreting the results to determine if the patient has a medical condition and means for providing a diagnosis based on the interpretation of the results by an individual or a healthcare professional by generating a report. The report includes a diagnosis of the presence of a microbiome disease and/or a treatment recommendation based on the results of said tests.

Further, based on results of the tests performed may include automated data analysis algorithms to interpret test results, classify microbiome diseases, and generate comprehensive reports detailing the presence, abundance, and potential implications of detected microorganisms.

Further, the reports generated by the system may include actionable recommendations for clinicians or healthcare providers, such as treatment options, antimicrobial susceptibility profiles, or further diagnostic steps based on the identified microbiome diseases.

The system may incorporate a user-friendly interface for inputting patient data, viewing test results, and accessing generated reports, facilitating seamless integration into clinical workflows and decision-making processes.

The system could include computational algorithms for analyzing test results and generating reports based on established criteria for microbiome disease identification and assessment. Further, the results could be communicated back to individuals or healthcare providers through secure online portals or telecommunication channels.

The system may have a built-in interface for immediate result interpretation, providing instant feedback to healthcare providers or individuals, possibly supplemented with connectivity for remote consultation or data sharing.

The system could include decision support tools for guiding treatment decisions based on microbiome disease assessments, potentially integrating with electronic health records for comprehensive patient management.

The system may feature advanced data analytics capabilities, utilizing machine learning algorithms for pattern recognition and disease prediction, leading to more personalized and precise diagnostics.

### Detection and Assessment of Gut Microbiome Diseases

Example: A patient presents with symptoms of chronic diarrhea and abdominal pain. A stool sample is collected and analyzed using the described method. The tests reveal an overgrowth of pathogenic bacteria such as Clostridium difficile and reduced levels of beneficial bacteria like Bifidobacterium. Additionally, elevated levels of inflammatory markers and specific antibodies against C. difficile toxins are detected. Based on these findings, the report confirms a diagnosis of Clostridium difficile infection (CDI) and recommends antibiotic therapy along with probiotic supplementation to restore gut microbiota balance.

Detection and Assessment of Oral Microbiome Diseases Example: A patient complains of persistent bad breath and gum inflammation. A saliva sample is collected and analyzed using the described method. The tests reveal a high abundance of periodontal pathogens such as P. gingivalis and elevated levels of VSCs indicative of oral malodor. Additionally, specific antibodies against P. gingivalis are detected, suggesting an ongoing infection. Based on these findings, the report confirms a diagnosis of periodontal disease and recommends professional dental cleaning along with antimicrobial mouthwash to control the infection and improve oral hygiene.

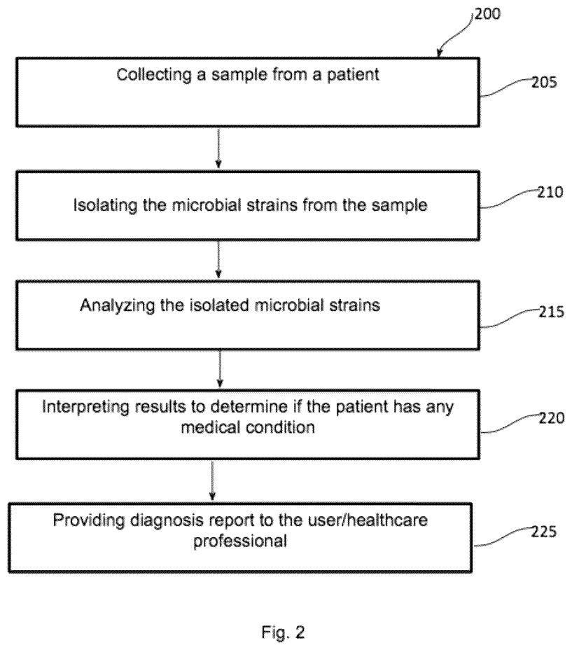

FIG. 2 is a flowchart illustrating a method for diagnosing medical conditions using microbiome diagnostics in accordance with an embodiment of the present invention. The method 200 starts at step 205.

At step 205, the biological sample containing microorganisms is collected from a patient. The sample is obtained from a biological source selected from the group consisting of blood, skin swab, urine, and feces.

At step 210, microbial strains are isolated from the sample by placing the sample on a culture medium and incubating;

At step 215, the isolated microbial strains are analyzed to detect the presence of certain pathogens using polymerase chain reaction (PCR) or other suitable analytic methods to detect the presence of pathogens including bacteria, viruses, fungi, or other microbes.

At step 220, the results are interpreted to determine if the patient has a medical condition.

At step 225, a diagnosis is provided a report based on the interpretation of the results to an individual or a healthcare professional.

Any of the methods, compositions, kits, and systems described herein can be used to detect and assess microbial diseases status of a subject. Disease status can include such information as a poor clinical outcome, good clinical outcome, high risk of disease, low risk of disease, complete response, partial response, stable disease, non-response, and recommended treatments for disease management.

The methods, compositions, systems and kits described herein provide assessments of microbial diseases as described herein, based on detection or measurement of one or more microbes in a biological sample or a microbiome obtained from a subject. In some applications, the biological sample is collected from a healthy subject. In some applications, the biological sample is collected from a subject with a disease. Indications that can be used with the methods, compositions, systems and kits as described herein include, but are not limited to IBD, preterm labor, obesity, diabetic foot ulcers, bacteremia, acne, infantile colic, type 2 diabetes, C. difficile, IBS, asthma, autism, psoriasis, allergies, cardiovascular disease, cancer, depression, cystic fibrosis, multiple sclerosis, urinary tract infection, radiation enteropathy, drug metabolism, chronic fatigue, type 1 diabetes, halitosis, and tooth decay.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A method for diagnosing medical conditions using microbiome diagnostics, comprising:
a. Collecting a sample from a patient, wherein the sample is obtained from a biological source selected from the group consisting of blood, skin swab, urine, and feces;
b. Isolating microbial strains from the sample by placing the sample on a culture medium and incubating;
c. Analyzing the isolated microbial strains to detect the presence of certain pathogens using polymerase chain reaction (PCR) or other suitable analytic methods to detect the presence of pathogens including bacteria, viruses, fungi, or other microbes;
d. Interpreting the results to determine if the patient has a medical condition; and
e. Providing a diagnosis based on the interpretation of the results to an individual or a healthcare professional.

2. The method as claimed in claim 1, wherein the sample is a bodily fluid and is selected from a group consisting of saliva, urine, stool, or tissue samples from organs or other body parts.

3. The method as claimed in claim 1, wherein the method also comprises a step of performing a sequencing test that sequences an entire genome or a portion of a genome of the microorganism to identify the presence of specific genes associated with a microbiome disease.

4. The method as claimed in claim 1, wherein the PCR-based test amplifies a specific region of the microorganism's genome to detect the presence of specific DNA sequences associated with a microbiome disease.

5. The method as claimed in claim 1, wherein the method includes a step of measuring the presence of specific antibodies associated with a microbiome disease.

6. The method as claimed in claim 7, wherein presence of specific antibodies associated with the microbiome disease is measured by ELISA-based test.

7. The method as claimed in claim 1, wherein the method further includes measuring presence of specific metabolites associated with the microbiome disease.

8. The method as claimed in claim 8, wherein the presence of specific metabolites associated with the microbiome disease is measured by mass spectrometry-based test measures

9. The method of claim 1, wherein the report includes a diagnosis of the presence of a microbiome disease and/or a treatment recommendation based on the results of said tests.

10. A system for diagnosing medical conditions using microbiome diagnostics, comprising:
a. a means for collecting a sample from a patient, wherein the sample is obtained from a biological source selected from the group consisting of blood, skin swab, urine, and feces;
b. a means for isolating microbial strains from the sample by placing the sample on a culture medium and incubating;
C. a means for analyzing the isolated microbial strains to detect the presence of certain pathogens using polymerase chain reaction (PCR) or other suitable analytic methods to detect the presence of pathogens including bacteria, viruses, fungi, or other microbes;
d. a means for interpreting the results to determine if the patient has a medical condition, and
e. a means for providing a diagnosis based on the interpretation of the results by an individual or a healthcare professional.

11. The system as claimed in claim 10, wherein the sample is a bodily fluid and is selected from a group consisting of saliva, urine, stool, or tissue samples from organs or other body parts.

12. The system as claimed in claim 10, wherein the PCR-based test amplifies a specific region of the microorganism's genome to detect the presence of specific DNA sequences associated with a microbiome disease.

13. The system as claimed in claim 10, wherein presence of specific antibodies associated with the microbiome disease is measured by ELISA-based test.

14. The system as claimed in claim 10, wherein the presence of specific metabolites associated with the microbiome disease is measured by mass spectrometry-based test measures

15. The system of claim 10, wherein the report includes a diagnosis of the presence of a microbiome disease and/or a treatment recommendation based on the results of said tests.
